(19) United States
(12) Patent Application Publication
(10) Pub. No.: US 2009/0282624 A1
(43) Pub. Date: Nov. 19, 2009

(54) Composition comprising a hydrophobic dye and an alkylene carbonate or a lactone and dyeing of keratinous fibres

(76) Inventor: Maxime De Boni, Tokyo (JP)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/394,624

(22) Filed: Feb. 27, 2009

Related U.S. Application Data
(60) Provisional application No. 61/064,594, filed on Mar. 14, 2008.

(30) Foreign Application Priority Data
Feb. 28, 2008 (FR) 0851300

(51) Int. Cl. A61K 8/49; A61Q 5/10

(52) U.S. Cl. 8/428

(57) ABSTRACT

A subject-matter of the present invention is compositions of use in the dyeing of human keratinous fibres, comprising, in a cosmetically acceptable medium, at least one dye having a hydrophobic nature and at least one compound chosen from those of following formula (I):

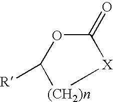

in which X represents O or CH₂, R′ represents a hydrogen, a C₁-C₈ alkyl or a C₁-C₄ hydroxyalkyl and n has a value of 1, 2 or 3. The compound of formula (I) can in particular be an alkylene carbonate or a lactone, the pH of the composition being between 2 and 6.

Another subject-matter of the invention is a method for dyeing keratinous fibres involving such a composition.

The subject-matter of the present invention is compositions of use in the dyeing of human keratinous fibres comprising, in a cosmetically acceptable medium, at least one hydrophobic dye and at least one compound chosen from alkylene carbonates and/or lactones.

Another subject matter of the invention is a process for dyeing human keratinous fibres employing such a composition.

Two main methods of dyeing human keratinous fibres and in particular the hair are known.

The first, known as oxidation or permanent dyeing, consists in employing one or more oxidation dye precursors, more particularly one or more oxidation bases, optionally in combination with one or more couplers.

Usually, oxidation bases are chosen from ortho- or para-phenylenediamines, ortho- or para-aminophenols and heterocyclic compounds. These oxidation bases are colourless or weakly coloured compounds which, in combination with oxidizing products, make it possible to access, by an oxidative coupling process, coloured entities which remain trapped inside the fibre.

Very often, the shades obtained with these oxidation bases can be varied by combining them with one or more couplers, the latter being chosen in particular from aromatic meta-diamines, meta-aminophenols, meta-diphenols and certain heterocyclic compounds, such as indole compounds.

The variety of the molecules involved as oxidation bases and couplers makes it possible to obtain a rich palette of colours.

The second dyeing method, known as direct or semi-permanent dyeing, comprises the application of direct dyes, which are coloured and colouring molecules having affinity for the fibres. Given the nature of the molecules employed, the latter remain rather at the surface of the fibre and penetrate inside the fibre only to a relatively slight extent in comparison with the small molecules of oxidation dye precursors.

The direct dyes generally employed are chosen from nitrobenzene, anthraquinone, nitropyridine, azo, methine, azomethine, xanthene, acridine, azine or triarylmethane direct dyes. The chemical entities employed can be nonionic, anionic (acid dyes) or cationic (basic dyes).

The majority of the direct dyes employed have a satisfactory solubility in aqueous media and there now exist numerous dyeing vehicles suitable for receiving them.

The situation is different in the case of dyes having a hydrophobic nature.

This is because, in order to efficiently colour keratinous fibres, some of these hydrophobic dyes have to be, in the majority of cases, employed in the presence of solvents, the role of which is to carry them into the fibre. Use is frequently made, among cosmetic solvents known for this effect, of aromatic solvents. Mention may more particularly be made of benzyl alcohol, benzyloxyethanol or phenoxyethanol.

Nevertheless, the presence of aromatic alcohols in these formulations magnifies the problem of staining of the scalp and skin encountered in dyeing.

In addition, and this represents another disadvantage in the use of such direct dyes, the presence of aromatic solvents, which are virtually insoluble in aqueous media, necessitates the use of large amounts of cosolvents (usually ethanol) in order to render them compatible with conventional dyeing formulations.

One of the objectives of the present invention is thus to provide dyeing compositions comprising at least one direct dye having a hydrophobic nature which exhibit satisfactory dyeing properties, in particular in that they make it possible to obtain powerful and homogeneous colourings between the tip and the root of the same fibre and from one fibre to another.

These objectives and others are achieved by the present invention, a subject-matter of which is dyeing compositions comprising, in a cosmetically acceptable medium, at least one or more hydrophobic direct dyes with a logP of greater than or equal to 2 and one or more compounds chosen from those of the following formula (I):

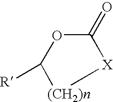

in which formula X represents an oxygen atom or a CH₂ group, R′ represents a hydrogen atom, a linear or branched C₁-C₈ alkyl radical or a linear or branched C₁-C₄ hydroxyalkyl radical and n has a value of 1, 2 or 3, the pH of the composition being between 2 and 6.

Another subject-matter of the invention is a method for dyeing human keratinous fibres, in particular the hair, which consists in applying the composition according to the invention to the said fibres.

Other characteristics and advantages of the invention will become more clearly apparent on reading the description and examples.

It should be noted that, in that which will follow and unless otherwise indicated, the limits of a range of values are included within this range.

As indicated above, the dyeing composition according to the invention comprises at least one direct dye having a hydrophobic nature with a logP of greater than or equal to 2.

The value of the logP conventionally represents the partition coefficient of the dye between octanol and water. The logP can be calculated according to the method described in the paper by Meylan and Howard, “Atom/Fragment Contribution Method for Estimating Octanol-Water Partition Coefficient”, J. Pharm. Sci., 84, 83-92, 1995. This value can also be calculated from many commercially available softwares which determine the logP as a function of the structure of a molecule. Mention may be made, by way of example, of the Epiwin software of the United States Environmental Protection Agency.

In particular, the dyes suitable for the implementation of the invention are chosen from the following compounds, alone or as mixtures:

| Dye | Chemical structure | logP |
|---|---|---|
| Disperse Red 17 | 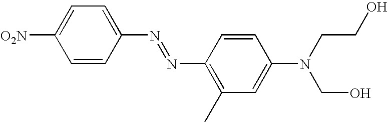 | 3.69 |
| Disperse Violet 1 | 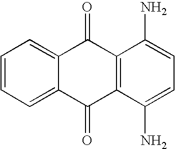 | 3.0 |
| HC Yellow 7 | 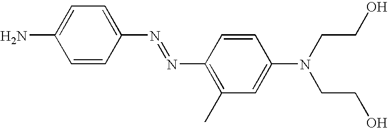 | 2.38 |
| Disperse Blue 377 | 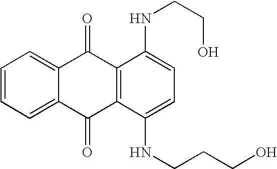 | 3.21 |
| Disperse Red 13 | 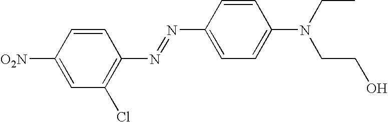 | 5.22 |
| Disperse Green 9 | 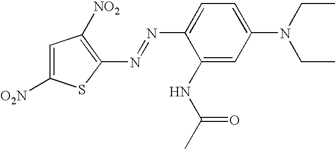 | 4.23 |
| Solvent Black 3 | 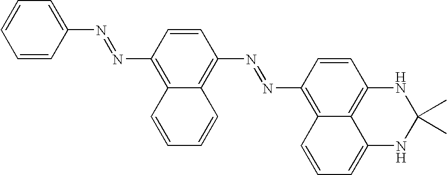 | 7.50 |
| Disperse Blue 143 | 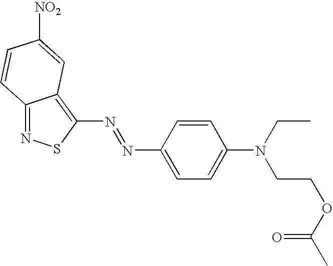 | 4.81 |
| Disperse Violet 63 | 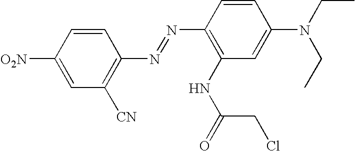 | 5.30 |
| Disperse Blue 60 | 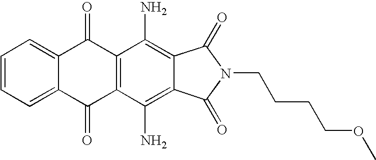 | 3.38 |
| Disperse Blue 14 | 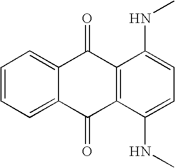 | 4.25 |
| Solvent Orange 15 | 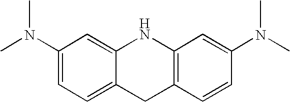 | 3.90 |
| Solvent Orange 7 | 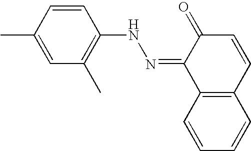 | 4.40 |
| Solvent Blue 14 | 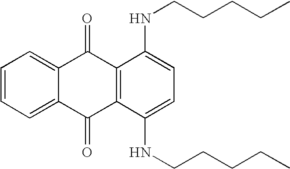 | 8.18 |
| Disperse Yellow 82 | 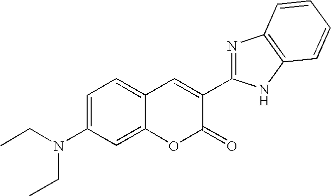 | 3.68 |

According to a specific embodiment of the invention, the content of hydrophobic direct dye(s) varies from 0.01 to 10% by weight, more particularly from 0.5 to 10% by weight, with respect to the weight of the composition, preferably from 0.01 to 5% by weight, with respect to the weight of the composition.

The dyeing composition according to the invention also comprises at least one compound chosen from those of abovementioned formula (I).

According to a first alternative form, the compound of formula (I) is such that X represents an oxygen atom and n has a value of 1.

Thus, the compound is an alkylene carbonate and corresponds to a compound of following formula (Ia):

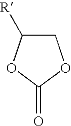

in which R′ represents a hydrogen atom, a linear or branched C₁-C₈ alkyl radical or a linear or branched C₁-C₄ hydroxyalkyl radical.

Preferably, the R′ radical represents a hydrogen atom, a linear or branched C₁-C₄ alkyl radical or a linear or branched C₁-C₂ hydroxyalkyl radical.

Mention may be made, as particularly advantageous examples of alkylene carbonate, of the compounds for which the R′ radical represents a hydrogen atom (corresponding to ethylene carbonate), a methyl group (corresponding to propylene carbonate) an ethyl group (corresponding to butylene carbonate) or a hydroxymethyl group (R′═—CH₂OH; corresponding to glyceryl carbonate).

Preferably, the alkylene carbonate employed is propylene carbonate.

According to a second alternative form of the invention, the compound of formula (I) is such that X represents a CH₂ group. In the context of this alternative form, n has a value of 1, 2 or 3.

In particular, the compound corresponds to a lactone of following formula (Ib):

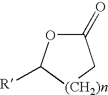

in which R′ represents a hydrogen atom, a linear or branched C₁-C₈ alkyl radical or a linear or branched C₁-C₄ hydroxyalkyl radical and n has a value of 1, 2 or 3.

Preferably, the R′ radical represents a hydrogen atom, a linear or branched C₁-C₆ alkyl radical or a linear or branched C₁-C₂ hydroxyalkyl radical.

According to a first embodiment, the lactone or lactones are such that the R′ radical represents a methyl group (corresponding to γ-valerolactone), an ethyl group (corresponding to γ-caprolactone), an n-propyl group (corresponding to γ-heptanolactone), an n-butyl group (corresponding to γ-octanolactone) or a hydroxymethyl group (R′═—CH₂OH; corresponding to dihydro-5-hydroxymethyl-2(3H)-furanone), with n having a value of 1.

According to a second alternative form, the lactone or lactones are such that the R′ radical represents a hydrogen atom (corresponding to δ-valerolactone) or an n-butyl group (corresponding to δ-nonanolactone), with n having a value of 2.

According to a third alternative form, the lactone or lactones are such that the R′ radical represents a hydrogen atom with n having a value of 3, corresponding to ε-caprolactone.

Preferably, the lactone of formula (Ib) is γ-caprolactone.

According to a particularly advantageous embodiment of the invention, the dyeing composition comprises one or more alkylene carbonates of formula (Ia) and preferably ethylene carbonate.

In accordance with a specific alternative form of the invention, the content of compound(s) of formula (I) represents from 0.5 to 50% by weight, with respect to the weight of the composition, and preferably from 1 to 30% by weight, with respect to the weight of the composition.

The composition according to the invention can also comprise one or more supplementary dyes. In particular, this or these supplementary dyes can be chosen from natural dyes, direct dyes other than dyes having a hydrophobic nature, oxidation dye precursors or their combinations.

The term “natural dyes” is to be understood to mean any dye or dye precursor which occurs naturally and which is produced either by extraction (and optionally purification) from a plant matrix or by chemical synthesis.

The natural dyes suitable in particular for the implementation of the invention are chosen from lawsone, juglone, alizarin, purpurin, carminic acid, kermesic acid, purpurogallin, protocatechualdehyde, indigo, isatin, curcumin, spinulosin, chlorophylls, chlorophyllins, orceins, hematein, hematoxylin, brazilin, brazilein, safflower dyes (such as, for example, carthamin), flavonoids (with, for example, morin, apigenidin or sandalwood), anthocyanins (of the apigeninidin type), carotenoids, tannins, sorghum and cochineal carmine.

Use may also be made of extracts or decoctions comprising these natural dyes and in particular henna-based extracts.

Preferably, the natural dye is chosen from lawsone, juglone, alizarin, purpurin, carminic acid, kermesic acid, purpurogallin, protocatechualdehyde, indigo, isatin, curcumin, spinulosin, apigenidin, chlorophyllin, sorghum, orceins and cochineal carmine.

The natural dyes, when they are present, represent from 0.01 to 10% by weight, with respect to the weight of the composition, preferably from 0.1 to 5% by weight, with respect to the weight of the composition.

The composition can also comprise one or more additional direct dyes other than the dyes having a hydrophobic nature described above which are chosen from ionic or nonionic entities, preferably cationic or nonionic entities.

Mention may be made, as examples of suitable additional direct dyes, of azo, methine, carbonyl, azine, nitro(hetero)aryl or tri(hetero)arylmethane direct dyes, porphyrins and phthalocyanines, alone or as mixtures.

More particularly, azo dyes comprise an —N═N-functional group, the two nitrogen atoms of which are not simultaneously participants in a ring. However, it is not out of the question for one of the two nitrogen atoms of the —N═N— sequence to be a participant in a ring.

Dyes of the family of the methines are more particularly compounds comprising at least one sequence chosen from >C═C< and —N═C<, the two atoms of which are not simultaneously participants in a ring. However, it is specified that one of the nitrogen or carbon atoms of these sequences can be a participant in a ring. More particularly, the dyes of this family result from compounds of the following types: methine, azomethine, mono- and diarylmethane, indoamines (or diphenylamines), indophenols, indoanilines, carbocyanines, azacarbocyanines and their isomers, diazacarbocyanines and their isomers, tetraazacarbocyanines or hemicyanines.

As regards dyes of the family of the carbonyls, mention may be made, for example, of dyes chosen from acridone, benzoquinone, anthraquinone, naphthoquinone, benzanthrone, anthranthrone, pyranthrone, pyrazolanthrone, pyrimidinoanthrone, flavanthrone, indanthrone, flavone, (iso)violanthrone, isoindolinone, benzimidazolone, isoquinolinone, anthrapyridone, pyrazoloquinazolone, perinone, quinacridone, quinophthalone, indigoid, thioindigo, naphthalimide, anthrapyrimidine, diketopyrrolopyrrole or coumarin dyes.

As regards dyes of the family of the cyclic azines, mention may in particular be made of azine, xanthene, thioxanthene, fluorindine, acridine, (di)oxazine, (di)thiazine or pyronine.

The nitro(hetero)aromatic dyes are more particularly nitrobenzene or nitropyridine direct dyes.

As regards the dyes of porphyrin or phthalocyanine type, use may be made of cationic or noncationic compounds optionally comprising one or more metals or metal ions, such as, for example, alkali and alkaline earth metals, zinc and silicon.

Mention may be made, as examples of additional synthetic direct dyes which are particularly suitable, of nitrobenzene dyes, azo, azomethine or methine direct dyes, azacarbocyanines, such as tetraazacarbocyanines (tetraazapentamethines), quinone and in particular anthraquinone, naphthoquinone or benzoquinone direct dyes or azine, xanthene, triarylmethane, indoamine, indigoid, phthalocyanine and porphyrin direct dyes, alone or as mixtures. More preferably still, these additional direct dyes are chosen from nitrobenzene dyes, azo, azomethine or methine direct dyes and tetraazacarbocyanines (tetraazapentamethines), alone or as mixtures.

These dyes can be monochromophoric dyes (that is to say, comprising only a single dye) or polychromophoric dyes, preferably di- or trichromophoric dyes, it being possible for the chromophores to be identical or different and from the same or different chemical family. It should be noted that a polychromophoric dye comprises several radicals, each resulting from a molecule which absorbs in the visible region between 400 and 800 nm. Furthermore, this absorbance of the dye requires neither preoxidation of the latter nor combination with other chemical entity(ies).

In the case of polychromophoric dyes, the chromophores are connected to one another by means of at least one connecting arm, which may or may not be cationic.

Mention may more particularly be made, among polychromophoric dyes, of symmetrical or asymmetrical di- or trichromophoric azo and/or azomethine (hydrazone) dyes comprising, on the one hand, at least one optionally fused 5- or 6-membered aromatic heterocycle comprising at least one quaternized nitrogen atom participating in the said heterocycle and optionally at least one other heteroatom (such as nitrogen, sulphur or oxygen) and, on the other hand, at least one optionally substituted phenyl or naphthyl group optionally carrying at least one OR group with R representing a hydrogen atom, an optionally substituted C₁-C₆ alkyl radical or an optionally substituted phenyl ring or at least one N(R′)₂ group with R′, which are identical or different, representing a hydrogen atom, an optionally substituted C₁-C₆ alkyl radical or an optionally substituted phenyl ring, it being possible for the R′ radicals to form, with the nitrogen atom to which they are bonded, a saturated 5- or 6-membered heterocycle, or alternatively either and/or both R′ radicals can form, each with the carbon atom of the aromatic ring in the ortho position with respect to the nitrogen atom, a saturated 5- or 6-membered heterocycle.

Mention may preferably be made, as cationic aromatic heterocycle, of 5- or 6-membered rings comprising from 1 to 3 nitrogen atoms, preferably 1 or 2 nitrogen atoms, one being quaternized, the said heterocycle furthermore optionally being fused with a benzene ring. It should likewise be noted that the heterocycle can optionally comprise another heteroatom other than nitrogen, such as sulphur or oxygen.

If the heterocycles or phenyl or naphthyl groups are substituted, they are substituted, for example, by one or more C₁-C₈ alkyl radicals optionally substituted by a hydroxyl group, a C₁-C₂ alkoxy group, a C₂-C₄ hydroxyalkoxy group, an acetylamino group, an amino group substituted by one or two C₁-C₄ alkyl radicals which optionally carry a hydroxyl group or it being possible for the two radicals to form, with the nitrogen atom to which they are attached, a 5- or 6-membered heterocycle optionally comprising another heteroatom identical to or different from nitrogen; a halogen atom; a hydroxyl group; a C₁-C₂ alkoxy radical; a C₂-C₄ hydroxyalkoxy radical; an amino radical; or an amino radical substituted by one or two identical or different C₁-C₄ alkyl radicals which optionally carry a hydroxyl group.

Preferably, the connecting arm is a linear, branched or cyclic C₁-C₂₀ alkyl chain which is optionally interrupted by at least one heteroatom (such as nitrogen or oxygen) and/or by at least one group comprising it (CO, SO₂), which is optionally interrupted by at least one substituted or unsubstituted phenyl or naphthyl group, which is optionally interrupted by at least one saturated, unsaturated or aromatic heterocycle which may or may not be fused with a phenyl ring, the said heterocycle comprising at least one quaternized nitrogen atom participating in the said ring and optionally at least one other heteroatom (such as oxygen, nitrogen or sulphur), and which is optionally interrupted by at least one quaternary ammonium group substituted by two C₁-C₁₅ alkyl groups, the connecting arm not comprising a nitro, nitroso or peroxo group.

If the heterocycles or aromatic nuclei are substituted, they are substituted, for example, by one or more C₁-C₈ alkyl radicals optionally substituted by a hydroxyl group, a C₁-C₂ alkoxy group, a C₂-C₄ hydroxyalkoxy group, an acetylamino group, an amino group substituted by one or two identical or different C₁-C₄ alkyl radicals which optionally carry a hydroxyl group or it being possible for the two radicals to form, with the nitrogen atom to which they are attached, a 5- or 6-membered heterocycle optionally comprising another heteroatom identical to or different from nitrogen; a halogen atom; a hydroxyl group; a C₁-C₂ alkoxy radical; a C₂-C₄ hydroxyalkoxy radical; an amino radical; or an amino radical substituted by one or two identical or different C₁-C₄ alkyl radicals which optionally carry a hydroxyl group.

The connection between the connecting arm and each chromophore is generally made by means of a heteroatom substituting the phenyl or naphthyl nucleus or by means of the quaternized nitrogen atom of the cationic heterocycle.

Mention may be made, among azo, azomethine, methine or tetraazapentamethine monochromophoric direct dyes which can be used according to the invention, of the cationic dyes described in Patent Applications WO 95/15144, WO 95/01772 and EP 714 954, FR 2 189 006, FR 2 285 851, FR 2 140 205, EP 1 378 544 and EP 1 674 073.

Thus, mention may very particularly be made of the cationic direct dyes corresponding to the following formula:

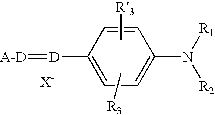

in which:
D represents a nitrogen atom or the —CH group,
R₁ and R₂, which are identical or different, represent a hydrogen atom; a C₁-C₄ alkyl radical which can be substituted by a —CN, —OH or —NH₂ radical or can form, with a carbon atom of the benzene ring, an optionally oxygen-comprising or nitrogen-comprising heterocycle which can be substituted by one or more C₁-C₄ alkyl radicals; or a 4′-aminophenyl radical,
R₃ and R′₃, which are identical or different, represent a hydrogen atom, a halogen atom chosen from chlorine, bromine, iodine and fluorine, a cyano radical, a C₁-C₄ alkyl radical, a C₁-C₄ alkoxy radical or an acetyloxy radical,
X⁻ represents an anion, preferably chosen from chloride, methyl sulphate and acetate,
A represents a group chosen from the following structures:

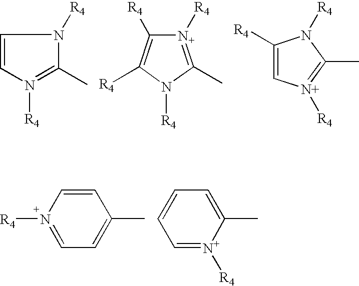

in which R₄ represents a C₁-C₄ alkyl radical which can be substituted by a hydroxyl radical;

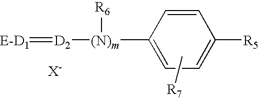

in which:
R₅ represents a hydrogen atom, a C₁-C₄ alkoxy radical or a halogen atom, such as bromine, chlorine, iodine or fluorine,
R₆ represents a hydrogen atom or a C₁-C₄ alkyl radical or forms, with a carbon atom of the benzene ring, a heterocycle which may comprise oxygen and/or may be substituted by one or more C₁-C₄ alkyl groups,
R₇ represents a hydrogen atom or a halogen atom, such as bromine, chlorine, iodine or fluorine,
D₁ and D₂, which are identical or different, represent a nitrogen atom or the —CH group,
m=0 or 1,
X⁻ represents a cosmetically acceptable anion preferably chosen from chloride, methyl sulphate and acetate,
E represents a group chosen from the following structures:

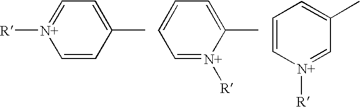

in which R′ represents a C₁-C₄ alkyl radical;
when m=0 and when D₁ represents a nitrogen atom, then E can also denote a group with the following structure:

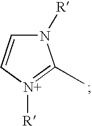

in which R′ represents a C₁-C₄ alkyl radical.

Use is very particularly made, among the abovementioned compounds, of the following compounds:

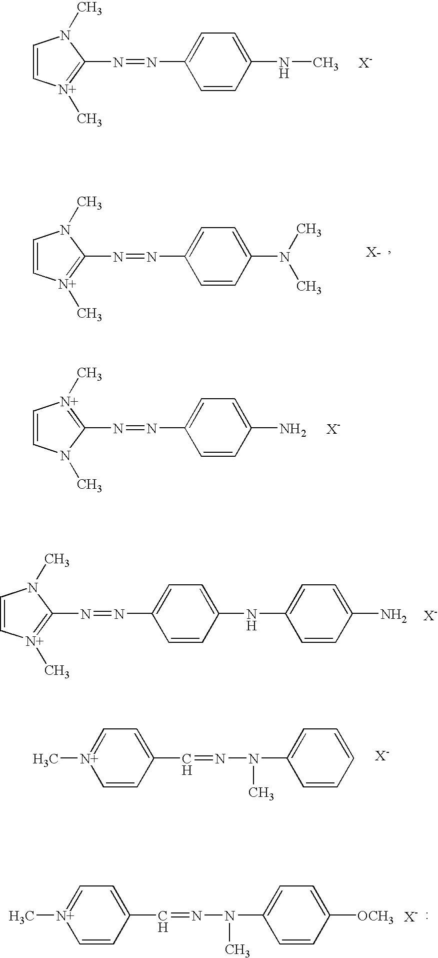

Mention may be made, among dyes of tetraazapentamethine type which can be used according to the invention, of the following compounds which appear in the table below:

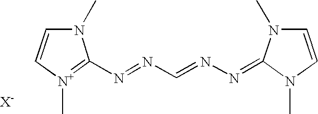

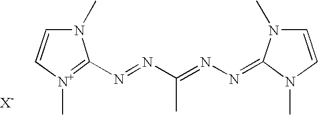

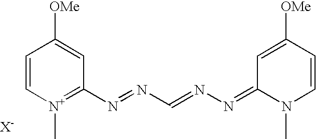

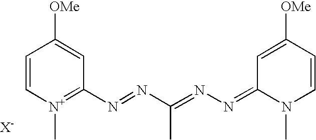

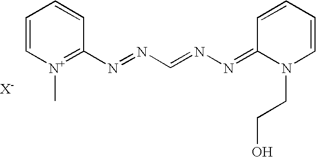

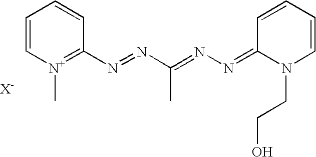

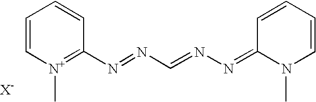

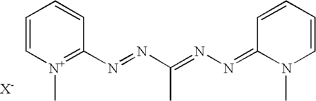

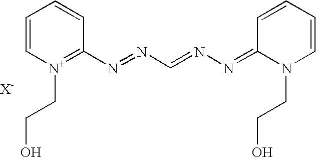

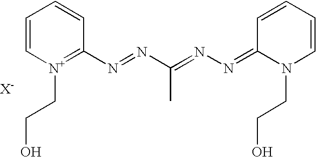

X⁻ represents an anion preferably chosen from chloride, iodide, methyl sulphate, ethyl sulphate or acetate.

When they are present, the synthetic additional direct dye or dyes represent from 0.0001 to 10% by weight, with respect to the weight of the composition, and preferably from 0.005 to 5% by weight, with respect to the same reference.

The dyeing composition can also comprise one or more oxidation dye precursors, more particularly one or more oxidation bases, optionally in combination with one or more couplers.

By way of example, the oxidation bases are chosen from para-phenylenediamines, bisphenylalkylenediamines, para-aminophenols, ortho-aminophenols, heterocyclic bases and their addition salts.

By way of example, the oxidation bases are chosen from para-phenylenediamines, bisphenylalkylenediamines, para-aminophenols, ortho-aminophenols, heterocyclic bases and their addition salts.

Mention may be made, among para-phenylenediamines, by way of example, of para-phenylenediamine, para-toluoylenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-N,N-bis(β-hydroxyethyl)amino-2-methylaniline, 4-N,N-bis(β-hydroxyethyl)amino-2-chloro-aniline, 2-(β-hydroxyethyl)-para-phenylenediamine, 2-fluoro-para-phenylenediamine, 2-isopropyl-para-phenylenediamine, N-(β-hydroxypropyl)-para-phenylene-diamine, 2-hydroxymethyl-para-phenylenediamine, N,N-dimethyl-3-methyl-para-phenylenediamine, N,N-(ethyl, β-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-(β-hydroxyethyloxy)-para-phenylene-diamine, 2-(β-acetylaminoethyloxy)-para-phenylene-diamine, N—(β-methoxyethyl)-para-phenylenediamine, 4-aminophenylpyrrolidine, 2-thienyl-para-phenylene-diamine, 2-(β-hydroxyethylamino)-5-aminotoluene, 3-hydroxy-1-(4′-aminophenyl)pyrrolidine, and their addition salts with an acid.

Among the para-phenylenediamines mentioned above, para-phenylenediamine, para-toluoylenediamine, 2-isopropyl-para-phenylenediamine, 2-(β-hydroxyethyl)-para-phenylenediamine, 2-(β-hydroxyethyloxy)-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, 2-chloro-para-phenylenediamine, 2-(β-acetylaminoethyloxy)-para-phenylenediamine, and their addition salts with an acid, are particularly preferred.

Mention may be made, among bisphenylalkylenediamines, by way of example, of N,N′-bis(β-hydroxy-ethyl)-N,N′-bis(4′-aminophenyl)-1,3-diaminopropanol, N,N′-bis(β-hydroxyethyl)-N,N′-bis(4′-aminophenyl)-ethylenediamine, N,N′-bis(4-aminophenyl)tetramethylene-diamine, N,N′-bis(β-hydroxyethyl)-N,N′-bis(4-aminophenyl)tetramethylenediamine, N,N′-bis(4-methyl-aminophenyl)tetramethylenediamine, N,N′-bis(ethyl)-N,N′-bis(4′-amino-3′-methylphenyl)ethylenediamine, 1,8-bis(2,5-diaminophenoxy)-3,6-dioxaoctane, and their addition salts.

Mention may be made, among para-aminophenols, by way of example, of para-aminophenol, 4-amino-3-methylphenol, 4-amino-3-fluorophenol, 4-amino-3-chlorophenol, 4-amino-3-(hydroxymethyl)phenol, 4-amino-2-methylphenol, 4-amino-2-(hydroxymethyl)phenol, 4-amino-2-(methoxymethyl)phenol, 4-amino-(2-aminomethyl)phenol, 4-amino-2-[(β-hydroxyethyl)amino-methyl]phenol, 4-amino-2-fluorophenol, and their addition salts with an acid.

Mention may be made, among ortho-aminophenols, by way of example, of 2-aminophenol, 2-amino-5-methylphenol, 2-amino-6-methylphenol, 5-acetamido-2-aminophenol, and their addition salts.

Mention may be made, among heterocyclic bases, by way of example, of pyridine derivatives, pyrimidine derivatives and pyrazole derivatives.

Mention may be made, among pyridine derivatives, of the compounds described, for example, in Patents GB 1 026 978 and GB 1 153 196, such as 2,5-diamino-pyridine, 2-(4-methoxyphenyl)amino-3-aminopyridine, 3,4-diaminopyridine, and their addition salts.

Other pyridine oxidation bases of use in the present invention are the oxidation bases 3-aminopyrazolo[1,5-a]pyridines or their addition salts described, for example, in Patent Application FR 2 801 308. Mention may be made, by way of example, of pyrazolo[1,5-a]pyridin-3-ylamine; 2-(acetylamino)-pyrazolo[1,5-a]pyridin-3-ylamine; 2-(morpholin-4-yl)pyrazolo[1,5-a]pyridin-3-ylamine; 3-aminopyrazolo-[1,5-a]pyridine-2-carboxylic acid; 2-methoxypyrazolo-[1,5-a]pyridin-3-ylamine; (3-aminopyrazolo[1,5-a]pyridin-7-yl)methanol; 2-(3-aminopyrazolo[1,5-a]pyridin-5-yl)ethanol; 2-(3-aminopyrazolo[1,5-a]pyridin-7-yl)ethanol; (3-aminopyrazolo[1,5-a]pyridin-2-yl)methanol; 3,6-diaminopyrazolo[1,5-a]pyridine; 3,4-diaminopyrazolo[1,5-a]pyridine; pyrazolo[1,5-a]-pyridine-3,7-diamine; 7-(morpholin-4-yl)pyrazolo[1,5-a]pyridin-3-ylamine; pyrazolo[1,5-a]pyridine-3,5-diamine; 5-(morpholin-4-yl)pyrazolo[1,5-a]pyridin-3-ylamine; 2-[(3-aminopyrazolo[1,5-a]pyridin-5-yl)(2-hydroxyethyl)amino]ethanol; 2-[(3-aminopyrazolo[1,5-a]-pyridin-7-yl)(2-hydroxyethyl)amino]ethanol; 3-amino-pyrazolo[1,5-a]pyridin-5-ol; 3-aminopyrazolo[1,5-a]pyridin-4-ol; 3-aminopyrazolo[1,5-a]pyridin-6-ol; 3-aminopyrazolo[1,5-a]pyridin-7-ol; and their addition salts.

Mention may be made, among pyrimidine derivatives, of the compounds described, for example, in Patents DE 2 359 399, JP 88-169 571, JP 05-63 124 or EP 0 770 375 or Patent Application WO 96/15765, such as 2,4,5,6-tetraminopyrimidine, 4-hydroxy-2,5,6-triamino-pyrimidine, 2-hydroxy-4,5,6-triaminopyrimidine, 2,4-dihydroxy-5,6-diaminopyrimidine, 2,5,6-triamino-pyrimidine, and their addition salts and their tautomeric forms, when a tautomeric equilibrium exists.

Mention may be made, among pyrazole derivatives, of the compounds described in Patents DE 3 843 892 and DE 4 133 957 and Patent Applications WO 94/08969, WO 94/08970, FR-A-2 733 749 and DE 195 43 988, such as 4,5-diamino-1-methylpyrazole, 4,5-diamino-1-(β-hydroxyethyl)pyrazole, 3,4-diaminopyrazole, 4,5-diamino-1-(4′-chlorobenzyl)pyrazole, 4,5-diamino-1,3-dimethylpyrazole, 4,5-diamino-3-methyl-1-phenyl-pyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methyl-pyrazole, 4,5-diamino-1-(β-hydroxyethyl)-3-methyl-pyrazole, 4,5-diamino-1-ethyl-3-methylpyrazole, 4,5-diamino-1-ethyl-3-(4′-methoxyphenyl)pyrazole, 4,5-diamino-1-ethyl-3-(hydroxymethyl)pyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-aminoethyl)-amino-1,3-dimethylpyrazole, 3,4,5-triaminopyrazole, 1-methyl-3,4,5-triaminopyrazole, 3,5-diamino-1-methyl-4-(methylamino)pyrazole, 3,5-diamino-4-(β-hydroxyethyl)-amino-1-methylpyrazole, and their addition salts. Use may also be made of 4,5-diamino-1-(β-methoxyethyl)pyrazole.

Mention may also be made, as pyrazole derivatives, of diamino-N,N-dihydropyrazolopyrazolones, in particular those described in Application FR 2 886 136, such as the following compounds and their addition salts: 2,3-diamino-6,7-dihydro-1H,5H-pyrazolo[1,2-a]pyrazol-1-one, 2-amino-3-ethylamino-6,7-dihydro-1H,5H-pyrazolo[1,2-a]pyrazol-1-one, 2-amino-3-isopropylamino-6,7-dihydro-1H,5H-pyrazolo[1,2-a]pyrazol-1-one, 2-amino-3-(pyrrolidin-1-yl)-6,7-dihydro-1H,5H-pyrazolo[1,2-a]pyrazol-1-one, 4,5-diamino-1,2-dimethyl-1,2-dihydropyrazol-3-one, 4,5-diamino-1,2-diethyl-1,2-dihydropyrazol-3-one, 4,5-diamino-1,2-di(2-hydroxyethyl)-1,2-dihydropyrazol-3-one, 2-amino-3-(2-hydroxyethyl)amino-6,7-dihydro-1H,5H-pyrazolo[1,2-a]pyrazol-1-one, 2-amino-3-dimethylamino-6,7-dihydro-1H,5H-pyrazolo[1,2-a]pyrazol-1-one, 2,3-diamino-5,6,7,8-tetrahydro-1H,6H-pyridazino[1,2-a]pyrazol-1-one, 4-amino-1,2-diethyl-5-(pyrrolidin-1-yl)-1,2-dihydropyrazol-3-one, 4-amino-5-(3-(dimethylamino)pyrrolidin-1-yl)-1,2-diethyl-1,2-dihydropyrazol-3-one or 2,3-diamino-6-hydroxy-6,7-dihydro-1H,5H-pyrazolo[1,2-a]pyrazol-1-one.

Use will preferably be made, as heterocyclic bases, of 4,5-diamino-1-(β-hydroxyethyl)pyrazole and/or 2,3-diamino-6,7-dihydro-1H,5H-pyrazolo[1,2-a]pyrazol-1-one, and their addition salts.

The composition according to the invention can optionally comprise one or more couplers advantageously chosen from those conventionally used for the dyeing of keratinous fibres.

Mention may more particularly be made, among these couplers, of meta-phenylenediamines, meta-aminophenols, meta-diphenols, naphthalene couplers, heterocyclic couplers, and their addition salts.

Mention may be made, by way of examples, of 1,3-dihydroxybenzene, 1,3-dihydroxy-2-methylbenzene, 4-chloro-1,3-dihydroxybenzene, 2,4-diamino-1-(β-hydroxy-ethyloxy)benzene, 2-amino-4-(g-hydroxyethylamino)-1-methoxybenzene, 1,3-diaminobenzene, 1,3-bis(2,4-diaminophenoxy)propane, 3-ureidoaniline, 3-ureido-1-(dimethylamino)benzene, sesamol, 1-(β-hydroxyethyl-amino)-3,4-methylenedioxybenzene, α-naphthol, 2-methyl-1-naphthol, 6-hydroxyindole, 4-hydroxyindole, 4-hydroxy-N-methylindole, 2-amino-3-hydroxypyridine, 6-hydroxybenzomorpholine, 3,5-diamino-2,6-dimethoxy-pyridine, 1-N-(β-hydroxyethyl)amino-3,4-methylenedioxy-benzene, 2,6-bis(β-hydroxyethylamino)toluene, 6-hydroxyindoline, 2,6-dihydroxy-4-methylpyridine, 1H-3-methylpyrazol-5-one, 1-phenyl-3-methylpyrazol-5-one, 2,6-dimethylpyrazolo[1,5-b][1,2,4]triazole, 2,6-dimethyl[3,2-c][1,2,4]triazole, 6-methylpyrazolo[1,5-a]benzimidazole, their addition salts with an acid and their mixtures.

Generally, the addition salts of the oxidation bases and of the couplers which can be used in the context of the invention are chosen in particular from addition salts with an acid, such as hydrochlorides, hydrobromides, sulphates, citrates, succinates, tartrates, lactates, tosylates, benzenesulphonates, phosphates and acetates.

The oxidation base or bases, when they are present in the composition, advantageously represent from 0.0001 to 10% by weight, with respect to the weight of the composition, and preferably from 0.005 to 5% by weight, with respect to the weight of the composition.

The content of coupler(s), if it(they) is(are) present, advantageously represents from 0.0001 to 10% by weight, with respect to the weight of the composition, and preferably from 0.005 to 5% by weight, with respect to the weight of the composition.

The dyeing composition according to the invention can also comprise one or more conditioning agents.

Mention may be made, by way of example, of volatile or non-volatile and linear, cyclic, branched or unbranched silicones. These silicones can be provided in the form of oils, of resins or of gums. They can in particular be polyorganosiloxanes which are insoluble in the cosmetically acceptable medium.

Organopolysiloxanes are defined in more detail in the work by Walter Noll, “Chemistry and Technology of Silicones” (1968), Academic Press. They can be volatile or non-volatile.

When they are volatile, the silicones are chosen more particularly from those having a boiling point of between 60° C. and 260° C.

Use may also be made, as conditioning agent, of cationic polymers, such as polyquaterniums 22, 6, 10, 11, 35 and 37 and hexadimethrine chloride.

The concentration of conditioning agent(s) in the composition or compositions of use in the invention can vary from 0.01 to 10% by weight, with respect to the total weight of the composition, preferably from 0.05 to 5% by weight and more preferably still from 0.1 to 3% by weight.

The compositions according to the invention can additionally comprise one or more organic thickening agents.

The organic thickening agents can be chosen from fatty acid amides (coconut diethanol- or monoethanolamide, oxyethylenated alkyl ether carboxylic acid monoethanolamide), polymeric thickeners, such as cellulose thickeners (hydroxyethylcellulose, hydroxypropylcellulose, carboxymethylcellulose), guar gum and its derivatives (hydroxypropyl guar), gums of microbial origin (xanthan gum, scleroglucan gum), crosslinked homopolymers of acrylic acid or of acrylamidopropanesulphonic acid, and associative polymers (polymers comprising hydrophilic regions and fatty-chain hydrophobic regions capable, in an aqueous medium, of reversibly associating with one another or with other molecules).

According to a specific embodiment, the thickener is polymeric and is chosen from cellulose thickeners (hydroxyethylcellulose, hydroxypropylcellulose, carboxymethylcellulose), guar gum and its derivatives (hydroxypropyl guar), gums of microbial origin (xanthan gum, scleroglucan gum) or crosslinked homopolymers of acrylic acid or of acrylamidopropanesulphonic acid.

As regards the associative thickening agents, use may be made of one or more polymers of nonionic or ionic nature, preferably anionic or cationic nature.

Their chemical structure more particularly comprises at least one hydrophilic region and at least one hydrophobic region. The term “hydrophobic group” is to be understood to mean a radical or polymer having a saturated or unsaturated and linear or branched hydrocarbon chain comprising at least 8 carbon atoms, preferably from 10 to 30 carbon atoms, in particular from 12 to 30 carbon atoms and more preferably from 18 to 30 carbon atoms.

Preferably, the hydrocarbon group originates from a monofunctional compound.

By way of example, the hydrophobic group can result from a fatty alcohol, such as stearyl alcohol, dodecyl alcohol or decyl alcohol. It can also denote a hydrocarbon polymer, such as, for example, polybutadiene.

Mention may be made, among anionic amphiphilic polymers comprising at least one fatty chain (hydrophobic), of:
(I) polymers comprising at least one hydrophilic unit and at least one allyl ether unit having a fatty chain, more particularly those for which the hydrophilic unit is composed of an ethylenic unsaturated anionic monomer, advantageously of a vinylcarboxylic acid and very particularly of an acrylic acid or a methacrylic acid or the mixtures of these, and for which the allyl ether unit having a fatty chain corresponds to the monomer of following formula (A):

CH₂═CR′CH₂OBₙR (A)

in which R′ denotes H or CH₃, B denotes the ethyleneoxy radical, n is zero or denotes an integer ranging from 1 to 100 and R denotes a hydrocarbon radical chosen from alkyl, arylalkyl, aryl, alkylaryl or cycloalkyl radicals comprising from 8 to 30 carbon atoms, preferably from 10 to 24 carbon atoms and more particularly still from 12 to 18 carbon atoms. A more particularly preferred unit of formula (A) is a unit in which R′ denotes H, n is equal to 10 and R denotes a stearyl (C₁₈) radical.

Preference is given, among these anionic polymers having a fatty chain, to the polymers formed from 20 to 60% by weight of acrylic acid and/or methacrylic acid, from 5 to 60% by weight of lower alkyl (meth)acrylates, from 2 to 50% by weight of allyl ether having a fatty chain of formula (A) and from 0 to 1% by weight of a crosslinking agent which is a well known copolymerizable polyethylenic unsaturated monomer, such as diallyl phthalate, allyl (meth)acrylate, divinylbenzene, (poly)ethylene glycol dimethacrylate and methylenebisacrylamide.

Among the latter, preference is very particularly given to crosslinked terpolymers of methacrylic acid, of ethyl acrylate and of polyethylene glycol (10 EO) ether of stearyl alcohol (Steareth 10), in particular those sold by Allied Colloids under the names Salcare SC 80 and Salcare SC 90, which are 30% aqueous emulsions of a crosslinked terpolymer of methacrylic acid, of ethyl acrylate and of steareth-10 alkyl ether (40/50/10).
(II) the polymers comprising at least one hydrophilic unit of olefinic unsaturated carboxylic acid type and at least one hydrophobic unit of (C₁₀-C₃₀)alkyl ester of unsaturated carboxylic acid type.

Preferably, these polymers are chosen from those for which the hydrophilic unit of olefinic unsaturated carboxylic acid type corresponds to the monomer of following formula (B):

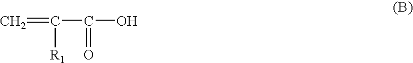

in which R₁ denotes H or CH₃ or C₂H₅, that is to say acrylic acid, methacrylic acid or ethacrylic acid units, and for which the hydrophobic unit of (C₁₀-C₃₀)alkyl ester of unsaturated carboxylic acid type corresponds to the monomer of following formula (C):

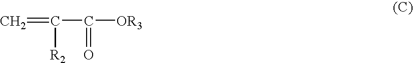

in which R₂ denotes H or CH₃ or C₂H₅ (that is to say, acrylate, methacrylate or ethacrylate units) and preferably H (acrylate units) or CH₃ (methacrylate units), R₃ denoting a C₁₀-C₃₀ and preferably C₁₂-C₂₂ alkyl radical.

The (C₁₀-C₃₀)alkyl esters of unsaturated carboxylic acids are, for example, lauryl acrylate, stearyl acrylate, decyl acrylate, isodecyl acrylate, dodecyl acrylate and the corresponding methacrylates, lauryl methacrylate, stearyl methacrylate, decyl methacrylate, isodecyl methacrylate and dodecyl methacrylate.

Use will more particularly be made, among anionic polymers having a fatty chain of this type, of polymers formed from a mixture of monomers comprising:

(i) essentially acrylic acid,
(ii) an ester of formula (C) described above in which R₂ denotes H or CH₃, R₃ denoting an alkyl radical having from 12 to 22 carbon atoms,
(iii) and a crosslinking agent which is a well known copolymerizable polyethylenic unsaturated monomer, such as diallyl phthalate, allyl (meth)acrylate, divinylbenzene, (poly)ethylene glycol dimethacrylate and methylenebisacrylamide.

Use will more particularly be made, among anionic polymers having a fatty chain of this type, of those composed of 95 to 60% by weight of acrylic acid (hydrophilic unit), of 4 to 40% by weight of C₁₀-C₃₀ alkyl acrylate (hydrophobic unit) and of 0 to 6% by weight of crosslinking polymerizable monomer or else those composed of 98 to 96% by weight of acrylic acid (hydrophilic unit), of 1 to 4% by weight of C₁₀-C₃₀ alkyl acrylate (hydrophobic unit) and of 0.1 to 0.6% by weight of crosslinking polymerizable monomer, such as those described above.

Preference is very particularly given, according to the present invention, among the above said polymers, to the products sold by Goodrich under the trade names Pemulen TR1, Pemulen TR2 or Carbopol 1382 and more preferably still to Pemulen TR1 and to the product sold by SEPPIC under the name Coatex SX.
(III) maleic anhydride/C₃₀-C₃₈ α-olefin/alkyl maleate terpolymers, such as the product (maleic anhydride/C₃₀-C₃₈ α-olefin/isopropyl maleate copolymer) sold under the name Performa V 1608 by Newphase Technologies.
(IV) acrylic terpolymers comprising:
   (a) from 20% to 70% by weight of a carboxylic acid having α,β-monoethylenic unsaturation,
   (b) from 20 to 80% by weight of a non-surface-active monomer having α,β-monoethylenic unsaturation other than (a),
   (c) from 0.5 to 60% by weight of a nonionic monourethane which is the reaction product of a monohydric surfactant with a monoisocyanate having monoethylenic unsaturation,
   such as those described in Patent Application EP-A-0 173 109, more particularly a methacrylic acid/methyl acrylate/dimethyl(meta-isopropenyl)benzyl isocyanate of ethoxylated behenyl alcohol (40 EO) terpolymer as a 25% aqueous dispersion.
(V) copolymers comprising, among their monomers, a carboxylic acid having α,β-monoethylenic unsaturation and an ester of a carboxylic acid having α,β-monoethylenic unsaturation and of an oxyalkylenated (C₈-C₃₀) fatty alcohol.

Preferably, these compounds also comprise, as monomer, an ester of a carboxylic acid having α,β-monoethylenic unsaturation and of a C₁-C₄ alcohol.

Mention may be made, as example of this type of compound, of Aculyn 22, sold by Röhm & Haas, which is a methacrylic acid/ethyl acrylate/oxyalkylenated stearyl methacrylate terpolymer.

The nonionic amphiphilic polymers having a fatty chain (hydrophobic) are preferably chosen from:
(1) celluloses modified by groups comprising at least one fatty chain, such as, in particular:
   hydroxyethylcelluloses modified by groups comprising at least one fatty chain, such as alkyl, arylalkyl or alkylaryl groups, or their mixtures, in which the alkyl groups are preferably C₈-C₂₂ alkyl groups, such as the product Natrosol Plus Grade 330 CS (C₁₆ alkyl groups), sold by Aqualon, or the product Bermocoll EHM 100, sold by Berol Nobel,
   those modified by polyalkylene glycol ether of alkylphenol groups, such as the product Amercell Polymer HM-1500 (polyethylene glycol (15) ether of nonylphenol), sold by Amerchol.
(2) hydroxypropyl guars modified by groups comprising at least one fatty chain, such as the product Esaflor HM 22 (C₂₋₂ alkyl chain), sold by Lamberti, or the products RE210-18 (C₁₄ alkyl chain) and RE205-1 (C₂₀ alkyl chain), sold by Rhône-Poulenc.
(3) copolymers of vinylpyrrolidone and of hydrophobic monomers having a fatty chain, with, for example:
   the products Antaron V216 or Ganex V216 (vinylpyrrolidone/hexadecene copolymer), sold by I.S.P.,
   the products Antaron V220 or Ganex V220 (vinylpyrrolidone/eicosene copolymer), sold by I.S.P.
(4) copolymers of C₁-C₆ alkyl methacrylates or acrylates and of amphiphilic monomers comprising at least one fatty chain, such as, for example, the methyl acrylate/oxyethylenated stearyl acrylate copolymer sold by Goldschmidt under the name Antil 208.
(5) copolymers of hydrophilic methacrylates or acrylates and of hydrophobic monomers comprising at least one fatty chain, such as, for example, the polyethylene glycol methacrylate/lauryl methacrylate copolymer.
(6) polymers having an aminoplast ether backbone possessing at least one fatty chain, such as the Pure Thix compounds provided by Sud-Chemie.
(7) polyether polyurethanes comprising, in their chain, both hydrophilic blocks of generally polyoxyethylene nature and hydrophobic blocks which can be aliphatic sequences alone and/or cycloaliphatic and/or aromatic sequences.

Preferably, the polyether polyurethanes comprise at least two hydrocarbon fatty chains, having from 8 to 30 carbon atoms, separated by a hydrophilic block, it being possible for the hydrocarbon chains to be pendant chains or chains at the end of the hydrophilic block.

Use may be made, as examples of nonionic polyether polyurethanes having a fatty chain which can be used in the invention, of Rheolate 205 having a urea functional group, sold by Rheox, or Rheolates 208, 204 or 212, and also Acrysol RM 184, Aculyn or Acrysol 44 and Aculyn or Acrysol 46 from Röhm & Haas [Aculyn 46 is a polycondensate of polyethylene glycol having 150 or 180 mol of ethylene oxide, of stearyl alcohol and of methylenebis(4-cyclohexyl isocyanate) (SMDI), at 15% by weight in a matrix of maltodextrin (4%) and of water (81%); Aculyn 44 is a polycondensate of polyethylene glycol having 150 or 180 mol of ethylene oxide, of decyl alcohol and of methylenebis(4-cyclohexyl isocyanate) (SMDI), at 35% by weight in a mixture of propylene glycol (39%) and of water (26%)].

Mention may also be made of the product Elfacos T210 having a C₁₂-C₁₄ alkyl chain and the product Elfacos T212 having a C₁₋₈ alkyl chain from Akzo and also the product DW 1206B from Röhm & Haas having a C₂₀ alkyl chain and having a urethane bond, provided at a dry matter content of 20% in water.

Use may also be made of solutions or dispersions of these polymers, in particular in water or in an aqueous/alcoholic medium. Mention may be made, as examples of such polymers, of Rheolate 255, Rheolate 278 and Rheolate 244, sold by Rheox. Use may also be made of the products DW 1206F and DW 1206J, provided by Röhm & Haas.

The polyether polyurethanes which can be used according to the invention are in particular those described in the paper by G. Fonnum, J. Bakke and Fk. Hansen, Colloid Polym. Sci., 271, 380-389 (1993).

The cationic amphiphilic polymers comprising at least one fatty chain (hydrophobic) used can be chosen in particular from quaternized cellulose derivatives, cationic polyurethanes or cationic polyvinyllactams and preferably from quaternized cellulose derivatives.

Mention may in particular be made, as examples of polymers of this type, of:
quaternized celluloses modified by groups comprising at least one fatty chain, such as alkyl, arylalkyl or alkylaryl groups comprising at least 8 carbon atoms, or mixtures of these groups,
quaternized hydroxyethylcelluloses modified by groups comprising at least one fatty chain, such as alkyl, arylalkyl or alkylaryl groups comprising at least 8 carbon atoms, or mixtures of these groups.

The alkyl radicals carried by the above quaternized celluloses or hydroxyethylcelluloses preferably comprise from 8 to 30 carbon atoms. The aryl radicals preferably denote the phenyl, benzyl, naphthyl or anthryl groups.

Examples of quaternized alkylhydroxyethyl-celluloses having C₈-C₃₀ fatty chains which may be pointed out include the products Quatrisoft LM 200, Quatrisoft LM-X 529-18-A, Quatrisoft LM-X 529-18B (C₁₋₂ alkyl) and Quatrisoft LM-X 529-8 (C₁₋₈ alkyl), sold by Amerchol, and the products Crodacel QM, Crodacel QL (C₁₂ alkyl) and Crodacel QS (C₁₈ alkyl), sold by Croda.

The content of thickened polymers, if they are present, usually varies from 0.05% to 5% by weight, with respect to the weight of the dyeing composition.

According to a particularly advantageous embodiment, the dyeing composition comprises one or more surface-active agents. The latter can be chosen without distinction, alone or as mixtures, from anionic, amphoteric, nonionic, zwitterionic and cationic surfactants.

Use is usually made, as regards anionic surfactants, of the salts, in particular the alkali metal salts, such as the sodium salts, the ammonium salts, the amine salts, the aminoalcohol salts or the alkaline earth metal salts, for example magnesium salts, of the following compounds, alone or as mixtures:
alkyl sulphates, alkyl ether sulphates, alkylamido ether sulphates or alkylaryl polyether sulphates;
alkylsulphonates, alkylamidesulphonates or alkylarylsulphonates;
alkyl sulphosuccinates, alkyl ether sulphosuccinates or alkylamide sulphosuccinates;
alkyl sulphoacetates;
acylsarcosinates; and acylglutamates;
alkyl esters of polyglycosidecarboxylic acids, such as alkyl glucosidecitrates, alkyl polyglycosidetartrates and alkyl polyglycosidesulphosuccinates;
alkyl sulphosuccinamates;
acyl isethionates or N-acyltaurates; or acyl lactylates;
alkyl-D-galactosideuronic acids;
polyoxyalkylenated alkyl ether carboxylic acids,
polyoxyalkylenated alkylaryl ether carboxylic acids or polyoxyalkylenated alkylamido ether carboxylic acids; the alkyl or acyl (RCO—) group of these compounds comprising from 10 to 24 carbon atoms and the aryl group preferably denoting a phenyl or benzyl group; the number of oxyalkylene and preferably oxyethylene groups is between 2 and 50.

As regards the nonionic surfactants, the latter can advantageously be chosen from the following compounds, alone or as mixtures:
polyethoxylated, polypropoxylated or polyglycerolated fatty alcohols,
polyethoxylated, polypropoxylated or polyglycerolated α-diols,
   the number of ethylene oxide or propylene oxide groups ranging from 2 to 50; the number of glycerol groups ranging from 2 to 30;
condensates of ethylene oxide and of propylene oxide with fatty alcohols;
polyethoxylated fatty amides having from 2 to 30 mol of ethylene oxide;
polyglycerolated fatty amides comprising from 1 to 5 glycerol groups;
polyethoxylated fatty amines having from 2 to 30 mol of ethylene oxide;
sorbitan ethoxylated fatty acid esters having from 2 to 30 mol of ethylene oxide;
sucrose fatty acid esters;
alkylpolyglucosides or N-alkylglucamine derivatives; these compounds comprising at least one alkyl or alkenyl chain comprising from 10 to 24 carbon atoms;
copolymers of ethylene oxide and of propylene oxide.

The cationic surfactants participating in the composition according to the invention can be chosen in particular from the following compounds, alone or as mixtures:
primary, secondary or tertiary fatty amines which are optionally polyethoxylated (2 to 30 mol of ethylene oxide) and their salts;
quaternary ammonium salts, such as tetraalkylammonium, alkylamidoalkyltrialkylammonium, trialkylbenzylammonium, trialkylhydroxyalkylammonium or alkylpyridinium chlorides or bromides;
alkylimidazoline derivatives; these compounds comprising at least one alkyl chain comprising from 10 to 24 carbon atoms.

Finally, the amphoteric surfactants can be chosen from the following compounds, alone or as mixtures:
derivatives of aliphatic secondary or tertiary amines in which the aliphatic group is a linear or branched chain comprising from 10 to 24 carbon atoms and comprising at least one water-solubilizing anionic group, such as, for example, a carboxylate, sulphonate, sulphate, phosphate or phosphonate group,
alkyl betaines, sulphobetaines, alkyl amido(C₆-C₈)alkyl betaines or alkyl amido(C₆-C₈)alkyl sulphobetaines,
   these compounds comprising at least one alkyl chain comprising from 10 to 24 carbon atoms.

Preferably, the surfactants are nonionic, anionic or amphoteric and more preferably still nonionic.

Usually, the surface-active agents represent an amount of between 0.01 and 50% by weight, preferably between 0.1 and 25% by weight, with respect to the weight of the composition.

The dyeing composition according to the invention can also include various adjuvants conventionally used in hair-dyeing compositions, such as, for example, anionic, nonionic, amphoteric or zwitterionic polymers other than the thickeners mentioned above, or their mixtures; inorganic thickening agents, such as, in particular, clays; antioxidants, such as, for example, ascorbic acid or erythorbic acid; reducing agents, with, inter alia, ammonium sulphite, ammonium bisulphite, ammonium metabisulphite or ammonium thiolactate; penetrating agents; sequestering agents, such as ethylenediaminetetraacetic acid or its salts; fragrances, mattifying agents, with, for example, titanium oxide; buffers; dispersing agents; film-forming agents; ceramides and preservatives.

The above adjuvants are generally present in amounts of, for each of them, between 0.01 and 20% by weight, with respect to the weight of the composition.

The cosmetically acceptable medium of the composition, which is a medium appropriate for the dyeing of human keratinous fibres, preferably comprises water and one or more solvents, including one or more compounds of formula (I) described in detail above.

The medium can optionally comprise one or more additional organic solvents other than the compounds of formula (I).

Mention may be made, as examples of such additional solvents, for example, of linear or branched C₂-C₄ alkanols, such as ethanol and isopropanol, polyols and polyol ethers, such as 2-butoxyethanol, hexylene glycol, propylene glycol, dipropylene glycol, propylene glycol monomethyl ether, diethylene glycol monoethyl ether, diethylene glycol monomethyl ether or glycerol, aromatic alcohols, such as benzyl alcohol, benzyloxyethanol or phenoxyethanol, and their mixtures.

The additional solvent or solvents can be present in proportions preferably ranging from 1 to 40% by weight, with respect to the total weight of the dyeing composition, and more preferably still from 5 to 30% by weight.

It should be noted that the amount of water in the composition according to the invention is preferably greater than 10% by weight, with respect to the weight of the composition, and more advantageously greater than or equal to 25% by weight. Preferably, the water content is between 25 and 98% by weight, with respect to the weight of the composition.

The pH of the composition according to the invention is between 2 and 6.

It can be adjusted to the desired value using one or more acidifying agents or one or more basifying agents normally used in the field.

Mention may be made, among acidifying agents, by way of example, of inorganic or organic acids, such as hydrochloric acid, orthophosphoric acid, sulphuric acid, carboxylic acids, such as acetic acid, tartaric acid, citric acid or lactic acid, or sulphonic acids.

Mention may be made, among basifying agents, by way of examples, of alkaline carbonates, alkanolamines, such as mono-, di- and triethanolamines and their derivatives, sodium hydroxide, potassium hydroxide and the compounds of following formula:

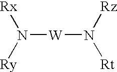

in which W is a propylene residue optionally substituted by a hydroxyl group or a C₁-C₆ alkyl radical and Rx, Ry, Rz and Rt, which are identical or different, represent a hydrogen atom or a C₁-C₆ alkyl or C₁-C₆ hydroxyalkyl radical.

The composition according to the invention can also comprise one or more oxidizing agents. The reference in this case is to a ready-for-use composition.

In particular, the ready-for-use composition is obtained by mixing at the time of use, before application, a composition described above with at least one composition comprising one or more oxidizing agents.

The oxidizing agent is preferably chosen from hydrogen peroxide, urea hydrogen peroxide, alkali metal bromates or ferricyanides, or peroxygenated salts, such as, for example, persulphates, perborates and percarbonates of alkali metals or alkaline earth metals, such as sodium, potassium or magnesium.

The use of hydrogen peroxide is particularly preferred.

This oxidizing agent is advantageously composed of hydrogen peroxide in aqueous solution (aqueous hydrogen peroxide solution), the strength of which can vary more particularly from 1 to 40 volumes and more preferably still from 5 to 40 volumes.

The compositions according to the invention can result from the mixing, at the time of use, of several compositions.

Another subject-matter of the invention is thus a method for dyeing keratinous fibres which consists in applying the composition described above.

In accordance with a first embodiment, the composition applied does not comprise an oxidizing agent. This embodiment is appropriate in the case where the composition does not comprise an oxidation dye precursor (base, coupler).

In accordance with a second embodiment, the composition is applied in the presence of at least one oxidizing agent.

This embodiment can be employed if the composition comprises, as dyes, only direct dyes (dye(s) having a hydrophobic nature and optionally one or more additional, synthetic and/or natural, direct dyes) or alternatively if the composition comprises one or more dyes having a hydrophobic nature and optionally one or more additional, synthetic and/or natural, direct dyes, in combination with one or more oxidation dye precursors (base/coupler).

According to a first alternative form of this embodiment, the ready-for-use composition which has just been described in detail and which is obtained by mixing at the time of use, before application, a composition according to the invention devoid of oxidizing agent with an oxidizing composition is applied to the fibres.

According to a second alternative form of this embodiment, the composition according to the invention devoid of an oxidizing agent and an oxidizing composition are applied successively and without intermediate rinsing.

The oxidizing composition employed comprises one or more oxidizing agents as defined above.

As regards the organic solvents optionally present in the oxidizing composition, reference may be made to the list indicated above in the context of the specifications of the composition according to the invention. It should be noted that these organic solvents can also be chosen from the compounds of formula (I).

Usually, the pH of the oxidizing composition is less than 7.

The oxidizing composition can be provided in the form of a solution, an emulsion or a gel.

It can optionally comprise one or more additives conventionally used in the field of the dyeing of human keratinous fibres, depending on the formulation form desired. Here again, reference may be made to the list of the additives which is given above.

Whatever the embodiment selected (with or without oxidizing agent), the mixture applied to the fibres is left in place for a period of time generally of the order of 1 minute to 1 hour, preferably of 10 minutes to 30 minutes.

The temperature during the method is conventionally between 10 and 200° C. and more particularly between ambient temperature (between 15 and 25° C.) and 80° C., preferably between ambient temperature and 60° C.

On conclusion of the treatment, the human keratinous fibres are optionally rinsed with water, washed with a shampoo, rinsed again with water and then dried or left to dry.

Concrete but non-limiting examples of the invention will now be presented.

### EXAMPLE 1

The following dyeing composition was prepared (contents expressed as grams of active material):

| | |
|---|---|
| HC Yellow 7 | 0.3 |
| Disperse Red 13 | 0.3 |
| Propylene carbonate | 25 |
| Ethanol | 6 |
| Hydroxyethyl cellulose (MW 1 300 000) | 1.5 |
| Citric acid | q.s. for pH 3 |
| Demineralized water | q.s. for 100 |

This composition was applied, on the one hand, to locks of natural grey hair comprising 90% of white hairs and, on the other hand, to locks of permed grey hair comprising 90% of white hairs, for 30 minutes at ambient temperature.

At the end of the leave-in time, the locks were rinsed, shampooed, then rinsed and dried. They were dyed a powerful and relatively unselective coppery shade.

### EXAMPLE 2

The following dyeing composition was prepared (contents expressed as grams of active material):

| | |
|---|---|
| Solvent Black 3 | 0.25 |
| Disperse Red 17 | 0.2 |
| Solvent Orange 7 | 0.1 |
| Propylene carbonate | 25 |
| Ethanol | 6 |
| Hydroxyethyl cellulose (MW 1 300 000) | 1.5 |
| Citric acid | q.s. for pH 3 |
| Demineralized water | q.s. for 100 |

This composition was applied, on the one hand, to locks of natural grey hair comprising 90% of white hairs and, on the other hand, to locks of permed grey hair comprising 90% of white hairs, for 30 minutes at ambient temperature.

At the end of the leave-in time, the locks were rinsed, shampooed, then rinsed and dried. They were dyed a relatively unselective brown shade.

### EXAMPLE 3

The following compositions were prepared (unless otherwise indicated, the proportions shown are percentage of active material):

| | Composition 1 | Composition 2 |
|---|---|---|
| | comparative | invention |
| Ethanol | 15 g % | 15 g % |
| Benzyl alcohol | 98 mmol % | — |
| Propylene carbonate | — | 98 mmol % |
| Disperse Red 13 | 0.2% | 0.2% |
| Water | q.s. for 100 | q.s. for 100 |

At the time of use, each of the compositions was applied to locks of natural hair comprising 90% of white hairs and to locks of permed hair comprising 90% of white hairs, in a proportion of 5 g of dyeing composition per gram of lock.

The leave-in time was 40 minutes at 40° C.

The locks were subsequently rinsed with water, then washed with Elseve Multivitamin 2-in-1 shampoo and dried.

### Evaluation of the Colouring:

The colouring is measured using the Minolta CM2600d spectrocolorimeter and while employing the CIE Lab system.

The selectivity, which demonstrates the homogeneity of the colouring obtained, is subsequently calculated using the following equation:

ΔE*=√(L*+a*+b*)

L*², a*² and b*² respectively representing the difference between the L*, a* and b* coefficients of the natural locks and of the permed locks.

The smaller the value of ΔE*, the better the selectivity of the colouring.

### Results:

| | ΔE* |
|---|---|
| Composition 1, comparative | 14.11 |
| Composition 2, according to the invention | 9.32 |

It is observed that the composition according to the invention makes it possible to obtain a colouring which is significantly more homogeneous than that resulting from the use of the comparative composition.

1. Dyeing composition comprising, in a cosmetically acceptable medium, at least one or more hydrophobic direct dyes with a logP of greater than or equal to 2 and one or more compounds chosen from those of the following formula: 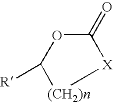
in which formula X represents an oxygen atom or a CH₂ group, R′ represent sa hydrogen atom, a linear or branched C₁-C₈ alkyl radical or a linear or branched C₁-C₄ hydroxyalkyl radical and n has a value of 1, 2, or 3, the pH of the composition being between 2 and 6.

2-13. (canceled)
